(19) United States
(12) Patent Application Publication
(10) Pub. No.: US 2002/0058060 A1
(43) Pub. Date: May 16, 2002

(54) Liposome for incorporating large amounts of hydrophobic substances

(76) Inventors: Pei Kan, Hsinchu (TW); Ae-June Wang, Hsinchu (TW); Won-Ko Chen, Hsinchu (TW); Chih-Wan Tsao, Pingtung City (TW)

Correspondence Address: VENABLE, Post Office Box 34385, Washington, DC, 20043-9998, US

(21) Appl. No.: 09/866,584

(22) Filed: May 30, 2001

(30) Foreign Application Priority Data
Sep. 25, 2000 (TW) 89119777

(51) Int. Cl. A61K 9/127

(52) U.S. Cl. 424/450

(57) ABSTRACT

A liposome for stably incorporating high content of hydrophobic substance is disclosed. The liposome includes two phospholipids with different phase transition temperatures such as saturated and unsaturated phosphatidyl cholines, hydrophobic substances, cholesterol, cholesterol derivatives, antioxidant and hydrophilic polymer-modified lipids such as MPEG-DSPE.

### BACKGROUND OF THE INVENTION

This application incorporates by reference Taiwanese application Serial No. 89119777, Filed Sep. 25, 2000.

1. Field of the Invention

The invention relates to the use of liposomes in the drug delivery system, and more particularly to liposomes capable of incorporating high content of hydrophobic drugs.

2. Description of the Related Art

Liposome technology has been exploited extensively for the purpose of drug delivery for many years. A typical liposome structure is composed of single or multiple layer membranes with hydrophobic domain between the phospholipid bilayers, and the interior aqueous compartment. Hydrophobic or hydrophilic compounds can be entrapped in the hydrophobic domain or encapsulated in the aqueous compartment, respectively. On the other hand, liposome can be constructed of natural constituents so that the liposome membrane is in principal identical to the lipid portion of natural cell membranes. It is considered that liposome is quite compatible with the human body when used as drug delivery system. In addition, liposome-based drug formulation also has been reported to be able to achieve the equivalent therapeutic efficacy to free drug, as well as reduce the systemic toxicity in many applications.

The hydrophobic drug, paclitaxel, was sold in the market in 1992, and used in phase II trials for treating breast and ovarian cancer. In 1998, it was used in a combination therapy with cisplatin for the treatment of non-small cell lung and ovarian cancer in phase I trials. However, due to its poor solubility in water, paclitaxel is prepared for clinical administration containing Cremophor EL® (polyethoxylated castor oil) and absolute ethanol in a 50/50 (vol/vol) ratio (Diluent 12). In clinical trials, a problem of anaphylactoid reaction, neutropenia, peripheral neuropathy, bradyarrhythmia and anemia was encountered. Meanwhile, the amount of cremophor EL necessary to solubilize the clinically required dose of paclitaxel is much higher than that administered with any other marketed drug. Cremophor vehicle thus is found to be responsible for hypersensitivity response. Premedication with corticosteroid, diphenhydramine or H₂ antagonist, and slow infusion of a large volume are needed to avoid the side effect. In contrast, owing to the aforementioned advantages of liposome-based drug delivery system, researches of incorporating paclitaxel in liposome for clinical paclitaxel administration have become a hot topic and been reported regularly.

Conventional paclitaxel-liposome was prepared at paclitaxel-lipid molar ratio of approximately 3 mole % regardless of whether the liposome is made of a mixture of phosphatidyl glycerol (PG) and phosphatidyl choline (PC) (U.S. Pat. No. 5,415,869; Sampedro, F et al., J Micrencapsul 11:309-318 (1993); Sharma, A. et al., Pharm Res 11:889-896 (1994); Shien, M. F. et al., J Ferm Bioeng 83:87-90 (1997)), or of unsaturated (U.S. Pat. No. 6,090,955; Bartoli, M. H. et al., J Micrencapsul 7:191-197 (1990); Riondel, J. et al., In Vivo 6:23-28 (1992); Sharma, D. et al., Melanoma Res 8:240-244 (1998)) or partially unsaturated PC (U.S. Pat. No. 5,683,715). At a drug/lipid ratio of 4 mole %, the paclitaxel-liposome system is stable only for 2 days while needle-like crystal precipitates appear during preparation at a drug/lipid ratio up to 8 mole % (Sathyamangalam, V et al., Biochemistry 33:8941-8947 (1994); Bernsdorff, C. et al., J Biomed Mater Res 46:141-149 (1999)). On the other hand, the liposome is prepared by employing hydrophilic polymer-conjugated phospholipid (methoxy polyethylene glycol-phosphatidyl ethanolamine) in order to enhance its circulation time in blood post iv administration (Crosasso, P. et al., J Control Release 63:19-30 (2000)). Liposomes with the prolonged circulation time in bloodstream makes it possible increasing the availability of the injected liposomes to reach the target cells before being metabolized. However, this formulation of the polymer-engrafted liposome with a maximal 3 mole % (paclitaxel/lipid ratio) quickly becomes unstable in one-week storage at 4° C.

Alternatively, a formulation of paclitaxel-liposome comprising a special phospholipid, cardiolipid, and phosphatidyl choline (PC) was disclosed in U.S. Pat. No. 5,424,073 and Int J Oncol 12:1035-1040 (Cabanes, A. et al., 1998). The molecular structure of cardiolipid is composed of one huge hydrophilic head and four aliphatic chains. The liposome prepared in accordance with this formulation increases the paclitaxel-lipid molar ratio to 9 mole %, however, it is stable only for 1 month when stored in liquid form at 4° C.

Generally, paclitaxel incorporated within the bilayer membrane of liposome is thermodynamically prone to self-aggregation, then precipitating from liposome. Previous researches have reported that the optimal paclitaxel/lipid molar ratio in a typical liposome formulation is ranged from 3 to 4 mole %, and paclitaxel-liposome is more stable when the drug/lipid ratio is kept at approximately 3 mole %. When the molar ratio is increased, needle-like crystal precipitates appear during the preparation process. Besides, it is known by person skilled in the art that drugs with a low drug/lipid ratio are commonly unsuitable for clinical administration. A high dose of liposome still may result in certain extent of toxicity due to the injection of excessive amounts of lipids in the body. Furthermore, increasing liposome concentration also raises the cost of production. Therefore, it is important to elevate the hydrophobic drug/lipid ratio in liposome-based drug delivery system by which the above drawbacks may be avoided.

### SUMMARY OF THE INVENTION

The objective of the invention, therefore, is to develop a liposome-based drug delivery system that is able to incorporate large amounts of hydrophobic compounds. Accordingly, the formulated liposome capable of incorporating high content of hydrophobic compound can maintain considerably stable for months, as well as reduce the possible side effects in the versatile applications.

According to the objective of the invention, there is provided a formulated liposome for incorporating large amounts of hydrophobic compounds. The composition of the liposome includes a first and second phospholipids, hydrophobic drugs, lipids modified by hydrophilic polymer (such as MPEG-DSPE), cholesterol, cholesterol derivatives, and antioxidants. The phase transition temperature of the first phospholipids, T_{g1}, is higher than that of the second phospholipids, T_{g2} while the drug delivery temperature T₁ and storage temperature T₂ are chosen at specified ranges subject to the order of T_{g1}>T₁>T₂>T_{g2}. This liposome composition results in coexitence of multiple incontinuous immiscible phases occurring on the bilayer membrane of liposome as the drug is delivered or stored.

The preferred phospholipids with high phase transition temperature are hydrogenated naturally-occurring phospholipids and saturated phospholipids with long carbon chain, such as phosphatidyl choline (PC), phosphatidyl glycerol (PG), phosphatidyl serine (PS), phosphatidyl ethanolamine (PE), phospatidyl inositol (PI), phosphaphatic acid (PA), or sphingomyelin (SM). Examples of hydrogenated phosphatidyl choline (PC) are hydrogenated egg phosphatidyl choline (HEPC) and hydrogenated soy phosphatidyl choline (HSPC), while examples of saturated phosphatidyl choline with long carbon chains are dipalmitoyl phosphatidyl choline (DPPC) and distearyloyl phosphatidyl choline (DSPC). The desired phospholipids may also be a combination of two or more PCs listed above. The lists of PC above are illustrations of specific phospholipids but are in no way intended to limit the scope thereof

The preferred phospholipids with low phase transition temperature are unsaturated phospholipids or saturated phospholipids with short carbon chains, such as phosphatidyl choline (PC), phosphatidyl glycerol (PG), phosphatidyl serine (PS), phosphatidyl ethanolamine (PE), phospatidyl inositol (PI), phosphaphatic acid (PA), or sphingomyelin (SM). Examples of synthetic or naturally-occurring unsaturated phosphatidyl choline (PC) are egg phosphatidyl choline (EPC) and soy phosphatidyl choline (SPC), while examples of synthetic or naturally-occurring unsaturated phosphatidyl choline and saturated phospholipids with short carbon chains are dimyristoyl phosphatidyl choline (DMPC) and dilauroyl phosphatidyl choline (DLPC). The desired phospholipids may also be a combination of two or more PC listed above. The list of PCs above are illustrations of specific phospholipids but are in no way intended to limit the scope thereof

These and other objects and features of the invention will become more fully apparent when the following detailed description of the invention is read in conjunction with the accompanying drawings

### BRIEF DESCRIPTION OF THE DRAWINGS

Other objects, features, and advantages of the invention will become apparent from the following detailed description of the preferred but non-limiting embodiments. The description is made with reference to the accompanying drawings in which:

FIG. 1 illustrates that coexisting phenomenon of two phases on the bilayer membrane of liposome resulting from the liposome composed of two phospholipids with very different phase transition temperatures (Korlach J. et al., Pro Natl Acad Sci USA, 96: 8461-8466 (1999)).

FIG. 2 illustrates that the incorporation efficiency and particle size of liposome are affected by altering cholesterol content according to a preferred embodiment of the invention.

Table 1 shows incorporation efficiency and particle size of different liposome compositions, according to a preferred embodiment of the invention.

Table 2 shows incorporation efficiency of liposome with different paclitaxel/lipid molar ratio, according to a preferred embodiment of the invention.

Table 3 shows incorporation efficiencies of the liposomes composed of only unsaturated or saturated phosphatidyl cholines at different drug/lipid molar ratios according to a preferred embodiment of the invention.

Table 4 shows the shelf stability of liposomes at different drug/lipid molar ratios according to a preferred embodiment of the invention.

Table 5 shows survival rate of mice received i.v. injections of either conventional paclitaxel (Cremophore EL/Ethanol=50/50 (v/v)) or paclitaxel-liposome at doses of 20 and 40 mg/kg, according to a preferred embodiment of the invention.

### DESCRIPTION OF THE PREFERRED EMBODIMENT

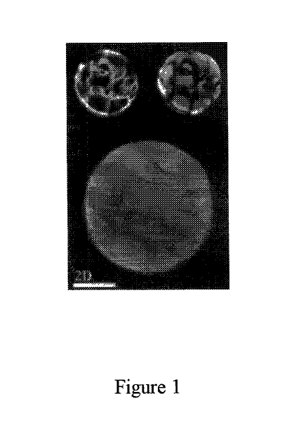

It has been observed and proved by Korlach J. et al. (96: 8461-8466, Pro Natl Acad Sci, USA, 1999) that at special ranges of two phospholipid combination and temperature, liposome composed of two phospholipids such as unsaturated and saturated phospholipids with different phase transition temperatures is able to form two separated phases, a gel phase and liquid-crystal phase, in the phospholipid bilayer, as shown in FIG. 1. The two immiscible phases coexist in the liposome and create several incontinuous regions.

It has been reported that hydrophobic compound such as paclitaxel has a tendency to undergo concentration-dependent aggregation in hydrophobic environments, forming intermolecular hydrogen bonds (Sathyamanglam, V et al., J. Pharm Sci 83: 1470-76 (1994)). Similarly, as a large amount of paclitaxel was embedded in the hydrophobic domain within bilayer membrane, it is thermodynamically prone to self-aggregating, destablizing the liposome. Accordingly, when the formulated liposome is prepared, two immiscible phases are formed and phase boundaries are speculated to construct a barrier stopping the self-aggregation process of hydrophobic molecules. As a result, a stable liposome capable of incorporating high content of hydrophobic compound becomes possible. The existence of lateral phase separated phospholipid regions is advantageous for incorporating large amount of hydrophobic molecules into the phospholipid bilayer. The formulated liposome can incorporate higher content of paclitaxel and remain more stable than any other liposome formulation ever reported.

The invention, hence, provides a liposome-based drug delivery system composing of two phospholipids with different phase transition temperatures. The phospholipids with high (T_{g1}) and low (T_{g2}) phase transition temperatures can be saturated and unsaturated phospholipids, respectively. The coexistence of several incontinuous immiscible two phases (e.g. gel phase and liquid-crystal phase) occurs in phospholpiid bilayer at a specific phospholipid composition and temperature (T), wherein T_{g1}>T>T_{g2}. The specific temperature T can be the liposome delivery or storage temperature, and selectively tuned depending on the properties of liposome system, and can even be a range that includes the liposome delivery and storage temperatures. For example, when the liposome delivery and storage temperatures are 37° C. and 4° C. respectively, the preferred phase transition temperatures of phospholipids are selected as T_{g1}>40° C. and T_{g2}<0° C.

The preferred phospholipids with high phase transition temperature are hydrogenated naturally-occurring phospholipids or saturated phospholipids with long carbon chains, such as phosphatidyl choline (PC), phosphatidyl glycerol (PG), phosphatidyl serine (PS), phosphatidyl ethanolamine (PE), phosphatidyl inositol (PI), phosphaphatic acid (PA), or sphingomyelin (SM). Examples of hydrogenated phosphatidyl choline (PC), which are not to be limitative, include hydrogenated egg phosphatidyl choline (HEPC) and hydrogenated soy phosphatidyl choline (HSPC). Examples of saturated phosphatidyl choline with long carbon chains include dipalmitoyl phosphatidyl choline (DPPC) and distearyloyl phosphatidyl choline (DSPC). The desired phospholipids may be a combination of two or more PCs listed above.

The preferred phospholipids with low phase transition temperature are unsaturated phospholipids or saturated phospholipids with short carbon chains, such as phosphatidyl choline (PC), phosphatidyl glycerol (PG), phosphatidyl serine (PS), phosphatidyl ethanolamine (PE), phosphatidyl inositol (PI), phosphaphatic acid (PA), or sphingomyelin (SM). Examples of unsaturated phosphatidyl choline (PC), which are not to be limitative, include egg phosphatidyl choline (EPC) and soy phosphatidyl choline (SPC). Examples of synthetic and naturally-occurring unsaturated phosphatidyl choline and saturated phospholipids with short carbon chains include dimyristoyl phosphatidyl choline (DMPC) and dilauroyl phosphatidyl choline (DLPC). The desired phospholipids may be a combination of two or more of the PCs listed above.

The following examples illustrate methods of preparing hydrophobic drugs/liposomes with saturated and unsaturated phosphatidyl choline (PC). The examples are intended to illustrate specific liposome compositions that include the phospholipids listed above, and methods of the invention, but are in no way intended to limit the scope thereof.

### EXAMPLE 1

### A Method of Preparing Paclitaxel-liposome

1.23 mg paclitaxel was added into the alcoholic admixture of 12.2 mg/ml egg phosphatidyl choline (EPC), 2.28 mg/ml hydrogenated egg phosphatidyl choline (HEPC), 2.28 mg/ml cholesterol and 5.4 mg/ml methoxy polyethylene glycol-distearyloyl phosphatidyl ethanolamine (MEG-DSPE) with a drug/lipid molar ratio of 1/14. The alcoholic admixture may also contain other antioxidants and cholesterol or cholesterol derivatives. Therefore, the composition of alcoholic admixture illustrated in this example was not to be limited. The solution was evaporated under vacuum to remove the solvent and form a lipid film on the wall of the round-bottom flask at which time, 1 ml, 10% (w/v) sucrose was added to the flask for hydration. Large multilamellar liposomes were suspended, followed by sonicating for 10 mins in order to obtain small unilamellar liposomes. Paclitaxel-containing liposomes were then sterilized by filtration through 0.2 μm cellulose acetate membrane. Laser particle size analyzer (Coulter N4 plus) was used to analyze the particle size. The average diameter was calculated to be approximately 120 nm. The concentration of the incorporated paclitaxel in liposome after filtration is determined by HPLC. It was approximately 1.0 mg/ml and the incorporation efficiency was about 80%.

### EXAMPLE 2

### A Method of Preparing Paclitaxel-liposome. Procedure is Similar to Example 1 in Addition to Extrusion Unit

Preparation of the liposome with the addition of extrusion unit was similar to that in the Example 1. Whereas, the liposomes obtained from sonication or hydration units were followed by extrusion with a series of membranes. Polycarbonate membranes with a uniform pore size ranging from 1.0 to 0.2 μm were used. The pressure from a nitrogen tank provided the driving force. The maximum pressure was set up to 750 psi according to the operation manual of the equipment manufactured by Lipex Co. The resultant liposome sample was sterilized by filtration, too. In this way, a narrow distribution of particle size of liposomes was obtained; the average particle size was estimated to be 150 nm with polydispersity index of 0.3. The loss of the incorporated paclitaxel during the process was about 10%.

### EXAMPLE 3

### Incorporation Efficiency and Particle Size Affected by Liposome Composition

Paclitaxel-liposome was prepared along with the procedure as described in Example 1. The liposomes based on different hydrogenated soy phosphatidyl choline (HSPC)/lipid ratios were prepared. The results in Table 1 indicated that increasing hydrogenated phosphatidyl choline content decreased paclitaxel incorporation efficiency. Precipitates appeared during preparation when HSPC/lipid ratio was raised to 60 mole %. Also, hydrogenated egg phosphatidyl choline (BEPC) could be used instead of hydrogenated soy phosphatidyl choline (HSPC). The resulting liposome exhibited high drug loading and small particle size comparable to previous liposomes with HSPC. It demonstrated that the hydrogenated phosphatidyl choline purified from distinct species was capable of carrying a high content of lipophilic drug too. But the optimal hydrogenated phosphatidyl choline/lipid ratio to attain the maximum incorporation efficiency depended on the combination of the selected phospholipids.

TABLE 1

| Liposome Composition | | Mean Particle Size |
|---|---|---|
| (molar ratio) | I.E. (%)^{#} | (nm) |
| Paclitaxel/EPC/HSPC/Chol/MPEG^{$} | 82.2 | 149.5 |
| (0.3/8/2/1/0.5) | | |
| Paclitaxel/EPC/HSPC/Chol/MPEG | 62.2 | 167.8 |
| (0.3/6/4/1/0.5) | | |
| Paclitaxel/EPC/HSPC/Chol/MPEG | Needle-like | |
| (0.3/4/6/1/0.5) | precipitates* | |
| Paclitaxel/EPC/HEPC/Chol/MPEG | 69.2 | 113.3 |
| (0.3/8/2/1/0.5) | | |
| Paclitaxel/EPC/HEPC/Chol/MPEG | 63.8 | 120.8 |
| (0.3/6/4/1/0.5) | | |
| Paclitaxel/EPC/HEPC/Chol/MPEG | 73.6 | 128.4 |
| (0.3/4/6/1/0.5) | | |

^{#}Incorporation efficiency = paclitaxel incorporated in liposome/paclitaxel added.
*Needle-like precipitates appeared during preparation.
^{$}MPEG means methoxy polyethylene glycol- distearyloyl phosphatidyl ethanolamine.

### EXAMPLE 4

### Incorporation Efficiency and Particle Size Affected by Varying the Cholesterol Content in Liposome Composition.

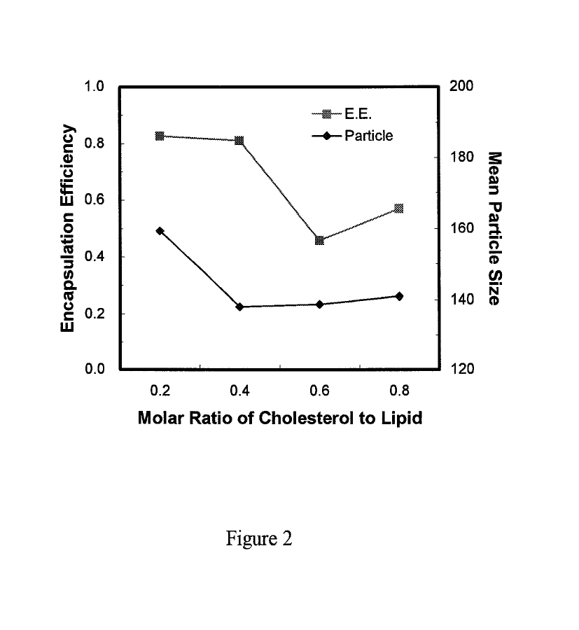

Varying cholesterol content affected the incorporation efficiency and particle size of liposome. On the other hand, incorporation of cholesterol may enhance the rigidity of liposome which is considered to be preferential due to prolong circulation time through i.v. administration. In the general formulation as shown in FIG. 2, increasing cholesterol content usually reduced the incorporation efficiency of paclitaxel as well as the average particle size. Cholesterol intercalated phospholipid bilayer where it was supposed to accommodate the lipophilic drug. Accordingly, the need for cholesterol addition was dependent on different combination of lipids and lipophilic drug. An optimal range of cholesterol content was necessary to achieve the desired incorporation efficiency and particle size. In FIG. 2 the range for paclitaxel-liposome system was indicated to be between 0.2 and 0.3 in term of cholesterol/lipid molar ratio.

### EXAMPLE 5

### Incorporation Efficiency Affected by Increasing Drug/Lipid Ratio in Liposome

Various aliquots of 10 mg/ml paclitaxel were added into the admixture to change the drug/lipid molar ratio in the liposome. Preparation of liposome was the same as Example 1. Incorporation efficiency of paclitaxel was calculated just after preparation according to the concentration determination by HPLC. It was found (Table 2) that incorporation efficiency was maintained above 80% as the drug/lipid molar ratio increases up to 20%. However, incorporation efficiency dropped to 60% when the drug/lipid ratio was increased to 25 mole %.

TABLE 2

| | | | | | Mean |
|---|---|---|---|---|---|
| | | | | | Particle |
| | [Lipid] | Drug/Lipid | [Paclitaxel] | I.E. | Size ± |
| Formulation | (mM) | (mole %) | (mg/ml) | (%) | SD (nm) |
| A* | 40 | 3 | 1.03 | 80.4 | 120.0 ± 45.5 |
| B^{#} | 20 | 7 | 1.04 | 84.5 | 114.3 ± 43.6 |
| C^{#} | 40 | 7 | 2.02 | 82.4 | 115.8 ± 41.0 |
| D^{#} | 20 | 10 | 1.34 | 78.8 | 116.2 ± 44.1 |
| E^{#} | 20 | 13 | 1.60 | 75.0 | 119.0 ± 44.2 |
| F^{#} | 20 | 15 | 2.07 | 81.0 | 125.4 ± 46.8 |
| G^{#} | 20 | 20 | 2.90 | 85.1 | 134.9 ± 44.6 |
| H^{#} | 20 | 25 | 2.32 | 54.6 | 146.3 ± 50.4 |

*Liposome composition EPC/HEPC/Chol/MPEG (32/8/12/2)
^{#}Liposome composition EPC/HEPC/Chol/MPEG (16/3/6/2)

### EXAMPLE 6

### Incorporation Efficiency Affected by Liposome Made of Either Unsaturated or Saturated PC Alone

The liposomes composed of only unsaturated or saturated phosphatidyl cholines were prepared as the same procedure in Example 1. The results were listed in Table 3 and showed that the liposome made of egg PC only was able to incorporate more than 90% paclitaxel when paclitaxel/lipid molar ratio was kept at 3 mole %. However, incorporation efficiency dropped to 40% once the molar ratio was raised up to 7 mole %. In contrast, the liposome made of only HEPC could not incorporate more than 3 mole % paclitaxel. The incorporation efficiency was estimated to be 40-60% when drug/lipid ratio was kept at 3 mole %.

TABLE 3

| | | | | | Mean |
|---|---|---|---|---|---|
| | | | | | Particle |
| Liposome | [Lipid] | Drug/Lipid | [Paclitaxel] | I.E. | Size ± |
| Composition | (mM) | (mole %) | (mg/ml) | (%) | SD (nm) |
| EPC/Chol/ | 20 | 3 | 0.45 | 88.4 | 142.9 ± 55.4 |
| MPEG-PE | | | | | |
| (20/8/1) | 20 | 7 | 0.52 | 42.1 | 174.1 ± 71.3 |
| HEPC/Chol/ | 20 | 3 | 0.35 | 68.1 | 93.2 ± 36.1 |
| MPEG-PE | | | | | |
| (10/1/1) | | | | | |
| HEPC/ | 60 | 3 | 1.20 | 34.0 | 118.9 ± 45.2 |
| DPPG/Chol | | | | | |
| (7/3/1) | | | | | |

### EXAMPLE 7

### Stability of Paclitaxel-liposome Stored at 4° C. Affected by Liposome Composition

Paclitaxel-liposome was stored at 4° C. immediately after preparation process. Paclitaxel crystals and large liposomes were removed by filtration through 0.2 μm CA-membrane. The concentration of paclitaxel was determined by HPLC. The results were listed in Table 4. At high paclitaxel/lipid ratio the formulated liposomes were considerably more stable than the liposome made of either egg PC or HEPC alone. As shown in Table 4, the changes in particle size and incorporation efficiency of the formulated liposome were less than 15% in one-month storage.

TABLE 4

| Liposome | Drug/Lipid | [Lipid] | [Paclitaxel]^{#} | I.E. (%)* | | |
|---|---|---|---|---|---|---|
| Composition | (mole %) | (mM) | (mg/ml) | 14 days | 30 days | 60 days |
| EPC/Chol/MPEG | 3 | 20 | 0.49 | 89.3 | 77.9 | |
| (20/8/1) | 7 | 20 | 0.45 | 67.8 | 35.4 | |
| HEPC/Chol/MPEG | 3 | 20 | 0.32 | 76.7 | 63.6 | |
| (10/1/1) | | | | | | |
| EPC/HEPC/Chol/MPEG | 3 | 40 | 0.77 | 108.4 | — | 73.9 |
| (32/8/12/2) | | | | | | |
| EPC/HEPC/Chol/MPEG | 7 | 20 | 0.92 | 105.3 | 97.9 | 104.6 |
| (16/3/6/2) | 7 | 40 | 2.02 | 104.4 | 97.3 | 105.0 |
| | 10 | 20 | 1.34 | — | 90.0 | 99.5 |
| | 13 | 20 | 1.60 | — | 93.7 | 98.5 |
| | 15 | 20 | 2.07 | 83.7 | 86.9 | 83.1 |
| | 15 | 20 | 2.07 | 94.2 | 109.7 | 86.0 |
| | 15 | 20 | 1.99 | 95.5 | 97.9 | 60.3 |
| | 20 | 20 | 2.90 | 88.7 | 91.7 | 85.5 |
| | 25 | 20 | 2.32 | — | 99.6 | 73.3 |

#Concentration of paclitaxel at day 0.
*Incorporation efficiency = [paclitaxel] at day N/[paclitaxel] at day 0.

### EXAMPLE 8

### The Stability of Paclitaxel/Liposome Stored with Other Methods

Paclitaxel-liposome was stored at −20 or −75° C. after the preparation process. Particle size and paclitaxel concentration were measured periodically. The results indicated that paclitaxel-liposome was stable at −75° C. for at least 3 months. Liposome could also be lyophilized, then stored in powder state at 4° C. for several months.

### EXAMPLE 9

### Large Amounts of a Hydrophobic Drug (ATRA) Encapsulated in the Liposome

2 mg all-trans retinoic acid (ATRA) was added into the alcoholic admixture of 12.2 mg/ml egg phosphatidyl choline (EPC), 2.28 mg/ml hydrogenated soy phosphatidyl choline (HSPC), 2.28 mg/ml cholesterol, and 5.4 mg/ml methoxy polyethylene glycol-distearyloyl phosphatidyl ethanolamine (MPEG-DSPE) according to a drug/lipid molar ratio of 1/3. The alcoholic admixture may also contain other antioxidants cholesterol, or cholesterol derivatives. Therefore, the composition of alcoholic admixture illustrated in this example was not to be limited. The solution was evaporated under vacuum to remove solvent and formed a lipid film on the wall of the round-bottom flask at which time 1 ml, 10% (w/v) sucrose was added to the flask for hydration. Large multi-lamellar liposomes were suspended, followed by sonicating for 10 mins to obtain small unilamellar liposomes. Retonic acid-containing liposome then was sterilized by filtration through 0.2 μm CA-membrane. Particle size was analyzed by laser particle size analyzer. The average diameter was calculated to be approximately 160 nm. Concentration of the incorporated retinoic acid in liposome after filtration was determined by HPLC, and it was approximately 1.9 mg/ml. The incorporation efficiency was more than 90%, and ATRA/lipid ratio was 33 mole %.

The liposome, prepared following the procedure of example 1, can encapsulate large amounts of ATRA. According to the result of example 9, the liposome prepared by description of example is able to encapsulate all of retinoic acid and its derivative, and the drug/lipid ratio is increased to 40 mole %, and people who skill in the art are able to use and make the same.

### EXAMPLE 10

### Large Amounts of Camptothecin (a Hydrophobic, Drug) Encapsulated in the Liposome

2 mg camptothecin was added into the admixture of 12.2 mg/ml egg phosphatidyl choline (EPC), 2.28 mg/ml hydrogenated egg phosphatidyl choline (HEPC), 2.28 mg/ml cholesterol and 5.4 mg/ml methoxy glycol-distearyloyl phosphatidyl ethanolamine (MPEG-DSPE) according to the drug/lipid molar ratio of 3/10. The solution was evaporated under vacuum to remove solvent and formed a lipid film on the wall of the round-bottom flask at which time 1 ml, 10% (w/v) sucrose was added to the flask for hydration. Large multilamellar liposomes were suspended, followed by sonicating for 10 minutes to obtain small unilamellar liposomes. Particle size was analyzed by laser particle size analyzer. The average diameter was calculated to be approximately 148 nm. No visible precipitate was found during preparation. The camptothecin/lipid ratio was 30 mole %.

The liposome, prepared following the procedure of example 1, can incorporate large amounts of camptothecin. According to the result of example 10, the liposome prepared by description of example is able to encapsulate all of camptothecin derivative, and the drug/lipid ratio is increased to 40 mole %, and people who skill in the art are able to use and make the same.

The examples 1, 2, 9 and 10 have indicated that the liposome prepared by the invention can incorporate large amounts of paclitaxel and its derivative, retinoic acid and its derivative, camptothecin and its derivative. In accordance with this aspect of the invention, the liposome, which is not limited to incorporate the compounds listed above, is capable of incorporating large amounts of paclitaxel and its derivative, retinoic acid and its derivative, camptothecin and its derivative, and mixture of combining two or more compounds listed above.

### EXAMPLE 11

### Toxicity of Conventional Paclitaxel and Liposomal Paclitaxel in Mice

Four groups of five to six-week-old male ICR mice received i.v. injections of either conventional paclitaxel (cremophore EL/ethanol=1/1) or paclitaxel-liposome at doses of 20 and 40 mg/kg. Survival rate in all the groups was recorded over 14 days, as listed in Table 5. Conventional paclitaxel formulation (Taxol®) with (50% cremophore EL/50% ethanol) exhibited marked toxicity as compared to liposomal paclitaxel.

TABLE 5

| | Dose (mg/kg) | Survival Rate |
|---|---|---|
| Conventional Paclitaxel | 20 | 4/5 |
| | 40 | 0/5 |
| Liposomal Paclitaxel | 40 | 8/8 |

### EXAMPLE 12

### Large Amounts of 5,5″-dihydroxymethyl-alpha-terthiophene(Polythiophene; a Hydrophobic Drug) Incorporated in the Liposome

1-2 mg 5,5″-dihydroxymethyl-alpha-terthiophene was added into the admixture of 12.2 mg/ml egg phosphatidyl choline (EPC), 2.28 mg/ml hydrogenated egg phosphatidyl choline (HEPC), 2.28 mg/ml cholesterol and 5.4 mg/ml methoxy polyethylene glycol-distearyloyl phosphatidyl ethanolamine (MPEG-DSPE). The solution was evaporated under vacuum to remove solvent and form a lipid film on the wall of the round-bottom flask at which time 1 ml, 10% (w/v) sucrose was added to the flask for hydration. Large multilamellar liposomes were suspended, followed by sonicating for 10 minutes to obtain small unilamellar liposomes. Particle size was analyzed by laser particle size analyzer. The average diameter was calculated to be approximately 124 nm. The concentration of 5,5″-dihydroxymethyl-alpha-terthiophene incorporated in liposome was estimated to be between 1.0-1.5 mg/mL. The 5,5″-dihydroxymethyl-alpha-terthiophene/lipid ratio was 50 mole %.

The liposome, prepared following the procedure of example 1, can incorporate large amounts of 5,5″-dihydroxymethyl-alpha-terthiophene. According to the result of example 12, the liposome prepared by description of example is able to encapsulate all of polythiophene derivative, and the drug/lipid ratio is increased to 100 mole %, and people who skill in the art are able to use and make the same.

According to the invention, the liposome can incorporate the hydrophobic compound such as paclitaxel in a drug/lipid ratio up to 20 mole %, and the variation of incorporation efficiency and particle size were within 15% stored at 4° C. for over 1 month. In the same condition of storage, the liposome with the drug/lipid ratio of 7 mole % was stable for over 2 months.

In comparison with the reported formulation of paclitaxel, the drug/lipid ratio was increased to 20 mole % by using the present liposome compositions of the invention. The dramatic improvement resulted from the composition of two phospholipids with different physical properties. It had been discovered that two phospholipids with different phase transition temperatures created immiscible two phases, and these incontinuous domains coexisting in the liposome could prevent paclitaxel from self-aggregation and precipitation (needle-like crystals). Even the liposome that contained large amounts of drugs could be maintained stable to a certain extent. In accordance with this theory, the formulation of liposome of the invention could be applied to incorporate other hydrophobic drugs that easily precipitated during the preparation or storage such as all-trans retinoic acid. The liposome of the invention increased the ATRA/lipid ratio to a maximum value of 33 mole % while conventional formulations of liposome had the ATRA/lipid ratio at a maximum of 20 mole %.

While the invention has been described by way of example and in terms of the preferred embodiment, it is to be understood that the invention is not limited to the disclosed embodiment. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A liposome for incorporating high content of hydrophobic substances comprising:
a first phospholipid;
a second phospholipid;
one or more hydrophobic substances; and
liposome-forming materials, wherein the first and the second phospholipid coexist in the liposome in a separated-phase form.

2. The liposome according to claim 1, wherein a phase transition temperature of the first phospholipid is higher than the specified temperatures of drug delivery and storage, while a phase transition temperature of the second phospholipid is lower than the specified temperatures of drug delivery and storage.

3. The liposome according to claim 2, wherein the first phospholipid is a hydrogenated naturally-occurring phospholipid or a saturated phospholipid with long carbon chains.

4. The liposome according to claim 3, wherein the first phospholipid is selected from the group consisting of phosphatidyl choline (PC), phosphatidyl glycerol (PG), phosphatidyl serine (PS), phosphatidyl ethanolamine (PE), phospatidyl inositol (PI), phosphaphatic acid (PA), and sphingomyelin (SM).

5. The liposome according to claim 4, wherein phosphatidyl choline (PC) is selected from the group consisting of hydrogenated egg phosphatidyl choline (HEPC), hydrogenated soy phosphatidyl choline (HSPC), dipalmitoyl phosphatidyl choline (DPPC) and distearyloyl phosphatidyl choline (DSPC),

6. The liposome according to claim 2, wherein the second phospholipid is an unsaturated phospholipid or a saturated phospholipid with short carbon chains.

7. The liposome according to claim 6, wherein the second phospholipid is selected from the group consisting of phosphatidyl choline (PC), phosphatidyl glycerol (PG), phosphatidyl serine (PS), phosphatidyl ethanolamine (PE), phospatidyl inositol (PI), phosphaphatic acid (PA), and sphingomyelin (SM).

8. The liposome according to claim 7, wherein phosphatidyl choline (PC) is selected from the group consisting of egg phosphatidyl choline (EPC), soy phosphatidyl choline (SPC), synthetic or naturally-occurring unsaturated phosphatidyl cholines, dimyristoyl phosphatidyl choline (DMPC) and dilauroyl phosphatidyl choline (DLPC).

9. The liposome according to claim 1, wherein the hydrophobic substances are one or more hydrophobic compounds.

10. The liposome according to claim 9, wherein the hydrophobic compound is paclitaxel and/or a derivative thereof.

11. The liposome according to claim 10, wherein paclitaxel and/or the derivative thereof are/is incorporated with a drug/lipid ratio ranging from about 0.5 mole % to 25 mole %.

12. The liposome according to claim 9, wherein the hydrophobic compound is retinoic acid and/or a derivative thereof.

13. The liposome according to claim 12, wherein the retinoic acid and/or the derivative thereof are/is incorporated with a drug/lipid ratio ranging from about 0.5 mole % to 40 mole %.

14. The liposome according to claim 9, wherein the hydrophobic compound is camptothecin and/or a derivative thereof.

15. The liposome according to claim 14, wherein the camptothecin and/or the derivative thereof are/is incorporated with a drug/lipid ratio ranging from about 0.5 mole % to 30 mole %.

16. The liposome according to claim 9, wherein the hydrophobic compound is polythiophene and/or a derivative thereof.

17. The liposome according to claim 16, wherein the polythiophene and/or the derivative thereof are/is incorporated with a drug/lipid ratio ranging from about 0.5 mole % to 100 mole %.

18. The liposome according to claim 9, wherein the hydrophobic drug is selected from the group consisting of paclitaxel and/or a derivatives thereof, retinoic acid and/or the derivatives thereof, camptothecin and/or the derivatives thereof, and polythiophene and/or derivatives thereof.

19. The liposome according to claim 1, wherein the liposome-forming materials are selected from the group consisting of hydrophilic polymer-modified lipids, cholesterol, cholesterol derivative, antioxidant, and mixtures thereof.

20. A liposome for incorporating high content of hydrophobic substances comprising:
a first phosphatidyl choline;
a second phosphatidyl choline;
one or more hydrophobic substances; and
liposome-forming materials, wherein the first and the second phosphatidyl cholines coexist in the liposome in a separated-phase form.

21. The liposome according to claim 20, wherein a phase transition temperature of the first phosphatidyl choline is higher than the specified temperatures of drug delivery and storage, while a phase transition temperature of the second phosphatidyl choline is lower than the specified temperatures of drug delivery and storage.

22. The liposome according to claim 21, wherein the first phosphatidyl choline is a hydrogenated naturally-occurring phosphatidyl choline or a saturated phosphatidyl choline with long carbon chains.

23. The liposome according to claim 22, wherein the phosphatidyl choline (PC) is selected from the group consisting of hydrogenated egg phosphatidyl choline (HBEPC), hydrogenated soy phosphatidyl choline (HSPC), dipalmitoyl phosphatidyl choline (DPPC) and distearyloyl phosphatidyl choline (DSPC),

24. The liposome according to claim 21, wherein the second phosphatidyl choline is an unsaturated phosphatidyl choline or a saturated phosphatidyl choline with short carbon chains.

25. The liposome according to claim 24, wherein phosphatidyl choline (PC) is selected from the group consisting of egg phosphatidyl choline (EPC), soy phosphatidyl choline (SPC), synthetic or natural-occurring unsaturated phosphatidyl cholines, dimyristoyl phosphatidyl choline (DMPC) and dilauroyl phosphatidyl choline (DLPC).

26. The liposome according to claim 20, wherein the hydrophobic substances are one or more hydrophobic drugs.

27. The liposome according to claim 26, wherein the hydrophobic drug is paclitaxel and/or a derivative thereof.

28. The liposome according to claim 27, wherein the paclitaxel and/or its derivative is incorporated with a drug/lipid ratio ranging from about 0.5 mole % to 25 mole %.

29. The liposome according to claim 26, wherein the hydrophobic drug is retinoic acid and/or a derivative thereof.

30. The liposome according to claim 29, wherein the retinoic acid and/or the derivative thereof are/is incorporated with a drug/lipid ratio ranging from about 0.5 mole % to 40 mole %.

31. The liposome according to claim 26, wherein the hydrophobic drug is camptothecin and/or a derivative.

32. The liposome according to claim 31, wherein the camptothecin and/or the derivative thereof are/is incorporated with a drug/lipid ratio ranging from about 0.5 mole % to 30 mole %.

33. The liposome according to claim 26, wherein the hydrophobic drug is polythiophene and/or a derivative thereof.

34. The liposome according to claim 33, wherein the polythiophene and/or the derivative thereof are/is incorporated with a drug/lipid ratio ranging from about 0.5 mole % to 100 mole %.

35. The liposome according to claim 26, wherein the hydrophobic drug is selected from the group consisting of paclitaxel, retinoic acid, camptothecin, polythiophene and their derivatives.

36. The liposome according to claim 20, wherein the liposome-forming materials are selected from the group consisting of hydrophilic polymer-modified lipids, cholesterol, cholesterol derivative, antioxidant, and mixture thereof.
